(19) United States
(12) Patent Application Publication
(10) Pub. No.: US 2022/0088344 A1
(43) Pub. Date: Mar. 24, 2022

(54) REMOTE INTERACTIVE CONTROL AND DELIVERY OF TACTILE BILATERAL STIMULATION (BLS)

(71) Applicant: LORI ELAINE PAPE, SUGAR LAND, TX (US)

(72) Inventor: LORI ELAINE PAPE, SUGAR LAND, TX (US)

(21) Appl. No.: 17/483,387

(22) Filed: Sep. 23, 2021

Related U.S. Application Data
(60) Provisional application No. 63/082,972, filed on Sep. 24, 2020.

(51) Int. Cl. A61M 21/00; G16H 40/67; G16H 20/40

(52) U.S. Cl. CPC A61M 21/00; G16H 40/67; A61M 2205/3584; A61M 2021/0022; A61M 2205/3553; G16H 20/40

(57) ABSTRACT

The present invention generally provides devices and the supporting software that allows a therapist to interactively control the delivery of tactile BLS to a remotely located client during a therapy session. The unique advantage of this invention is that the therapist and client can be in different physical locations during treatment with tactile BLS. The therapist's device supports configuring parameters such as the start, stop, and number of tactile stimulations delivered to the client. The client's device receives the parameters and activates the bilateral stimulation sequences accordingly. Each device connects to the Internet and communicates with a cloud-based service to exchange commands and settings. With this invention, the therapist and the client are no longer required to be in the same location during a treatment session incorporating tactile BLS.

### BACKGROUND OF THE INVENTION

The present invention relates to the field of psychotherapy, wherein a trained therapist delivers Bilateral Stimulation (BLS) to a client during a therapy session. Delivering BLS refers to the presentation of a visual, auditory, or tactile stimulus to one side of the body, then to the other side, followed by repeated sets of these alternating stimulations. A set is a sequence of one left-side vibration followed by one right-side vibration. One example of therapy where BLS is used is for Eye Movement Desensitization and Reprocessing (EMDR) treatment.

During treatment incorporating BLS, the therapist and client meet in person so the therapist can interactively guide the presentation of the BLS based on the observed behavior and responses by the client. A method for delivering tactile BLS might consist of a controller and a pair of tactile stimulators connected to the controller, with the limiting constraint that the therapist and client must be in close physical proximity to each other. The therapist holds the controller and selects parameters for the delivery of the tactile BLS sets. The client holds one of the tactile stimulators in each hand, which vibrate alternately as the therapist guides delivery of the BLS.

### SUMMARY OF THE INVENTION

The increasing trend in virtual or telehealth therapy sessions means that a therapist and a client may be remotely located, i.e., not occupying the same physical premises. The present invention provides a means to support tactile BLS when the client is at a different physical location than the therapist, allowing the therapist to interactively control the delivery of tactile BLS to a remotely located client during a therapy session.

The invention generally comprises: an internet enabled device to be used by the therapist, hereinafter referred to as the therapist device; an internet enabled device to be used by the client, hereinafter referred to as the client device; and a cloud service that supports authorization and secure connection between the devices. The client device includes tactile stimulators for the delivery of BLS. In the description that follows, these devices may be referred to as “Internet of Things (IoT)” devices, meaning any internet enabled device.

The therapist uses the therapist device to select parameters and control the delivery of the tactile BLS to the remotely located client. The client holds the tactile stimulators of the client device, which vibrate or otherwise deliver the stimulations during the session. As the session proceeds, the therapist guides and dynamically adjusts the stimulus delivery parameters based on the client's verbal and non-verbal responses, according to the treatment protocol. The unique advantage of this invention is that tactile BLS can be delivered even when the therapist and client are not at the same physical location.

### BRIEF DESCRIPTION OF THE DRAWINGS

FIG. 1 is a block diagram showing the components of one embodiment of the invention;

FIG. 2 illustrates components of the therapist IoT device, in accordance with one embodiment of the invention;

FIG. 3 illustrates components of the client IoT device, in accordance with one embodiment of the invention;

FIG. 4 shows the components of the IoT cloud service, in accordance with one embodiment of the invention; and

FIG. 5 shows an example of the user and device information maintained by the IoT cloud service in a secure database.

### DETAILED DESCRIPTION OF THE INVENTION

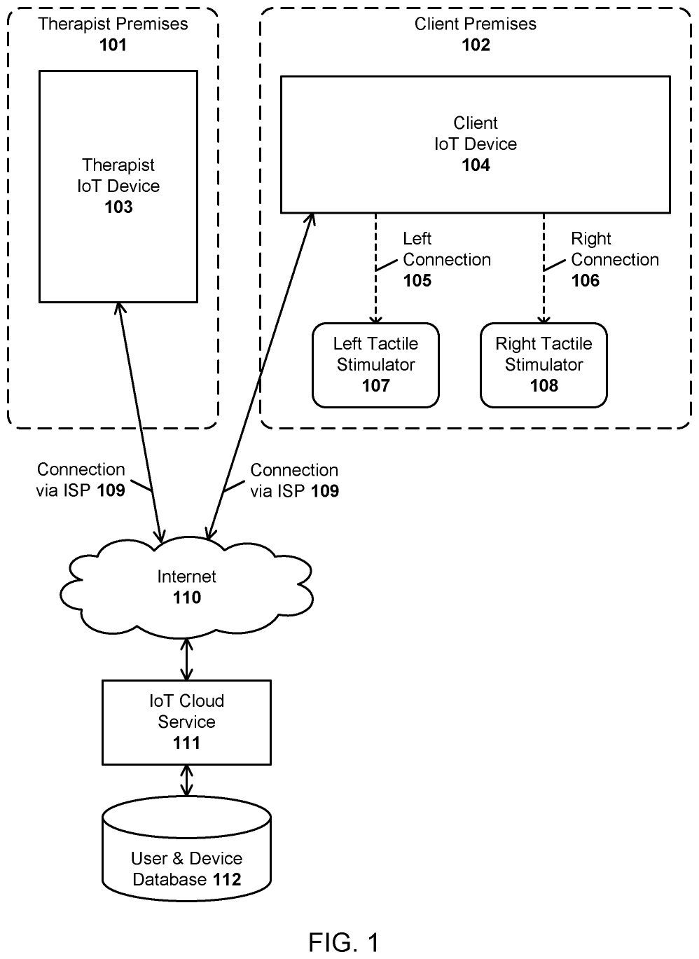

FIG. 1 provides an overview of one preferred embodiment of the invention. The therapist premises 101 and the client premises 102 are designated by the dotted lines encircling the therapist IoT device 103 and the client IoT device 104, respectively. The premises are shown in this way to illustrate that they may be physically separate, i.e., the therapist and client may be in different locations. However, the underlying principles of this invention also support the case where the therapist and client are in a single location, such as the therapist's office.

Each of the IoT devices 103 and 104 connect to the Internet 110 through a connection 109 with an Internet Service Provider (ISP). In some embodiments of the invention, the connection 109 is wireless, e.g., over Wi-Fi through a local router and modem, over a cellular connection such as 3G, 4G LTE, or similar. In other embodiments of the invention, the connection 109 may be wired to the local router or other networking communications device. Thereafter, the devices 103 and 104 connect to the IoT cloud service 111.

In one embodiment, the cloud service 111 may support, but is not limited to, the following functions:
a. Initial registration of each device 103 and 104, including adding the device and associated user (therapist) to a database 112;
b. Updates to the database 112 for adding or removing devices and users;
c. Security and authentication to ensure trusted communications with devices 103 and 104; and
d. Handling messaging between the two IoT devices 103 and 104.

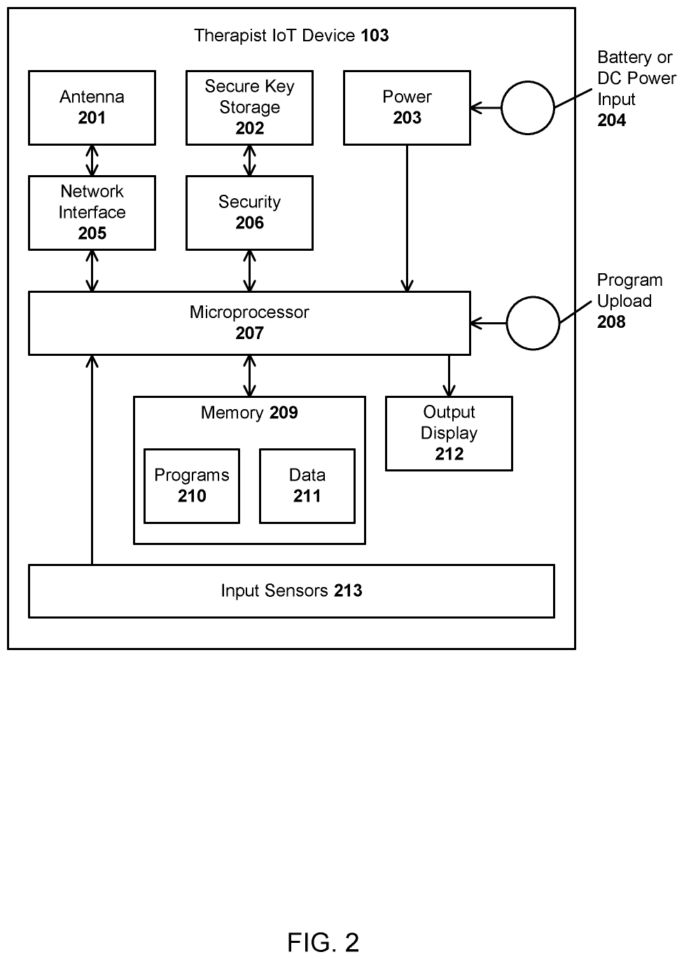

FIG. 2 illustrates the key components comprising one embodiment of a therapist IoT device 103. These components may include but are not limited to the following:
a. An antenna 201 and an associated network interface module 205;
b. A security module 206 and a mechanism for secure key storage 202;
c. A power management module 203, and a battery or DC power input 204 that provides power to the device;
d. A low-power microprocessor 207;
e. A program upload connection 208, such as a Universal Serial Bus (USB) micro-B connection or similar, to optionally support uploading program code and data;
f. Memory 209, such as static or dynamic memory, which provides the memory resource for both programs 210 and data 211;
g. An output display 212, which may be of various types commonly used in the industry, and is used for output indications such as the parameters, battery percentage remaining, connection status, and others; and
h. Input sensors 213, which are used to select the values of the various parameters related to tactile BLS.

In some embodiments, the input sensors 213 may include but are not limited to physical inputs such as knobs and switches, touchscreen devices for selections from a touchscreen display, and voice control for selecting options.

In some embodiments, the power management module 203 may handle recharging circuitry and logic to support DC power input 204 from a rechargeable battery, such as a Lithium Polymer (Li—Po) battery or other types of rechargeable batteries.

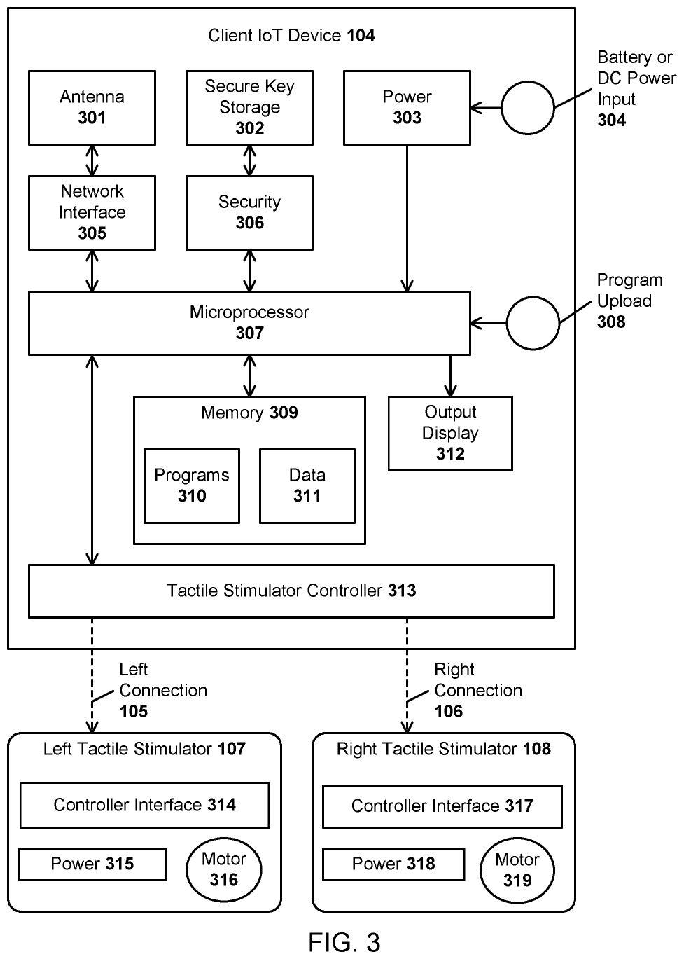

FIG. 3 illustrates the components of one embodiment of a client IoT device 104. These components may include but are not limited to the following:
a. An antenna 301 and an associated network interface module 305;
b. A security module 306 and a mechanism for secure key storage 302;
c. A power management module 303, and a battery or DC power input 304
that provides power to the device;
d. A low-power microprocessor 307;
e. A program upload connection 308, such as a USB micro-B connection to optionally support uploading program code and data; f Memory 309, such as static or dynamic memory, which provides the memory resource for both programs 310 and data 311;
g. Output display 312, which may optionally provide a visual display of the left and right stimulations and other status indications; and
h. A tactile stimulator controller 313, which issues commands and controls the left and right tactile stimulators 107 and 108 to produce the BLS.

In some embodiments, the output display 312 may include but is not limited to Thin-Film Transistor (TFT) Liquid Crystal Display (LCD) devices for displaying additional information such as connection status.

In some embodiments, the power management module 303 may handle recharging circuitry and logic to support DC power input 304 from a rechargeable battery, such as a Li—Po battery or other types of rechargeable batteries.

FIG. 3. also illustrates details of one embodiment of the left and right tactile stimulators 107 and 108. The left tactile stimulator 107 is connected to the device 104 by the left connection 105, and the right tactile stimulator 108 is connected by the right connection 106.

In some embodiments, the connections 105 and 106 may be wired, and in other embodiments they may be wireless, using industry standards such as Bluetooth Low Energy (BLE). This is indicated by the dotted line from the device 104 to the tactile stimulators 107 and 108.

In some embodiments, the left tactile stimulator 107 includes, but is not limited to, the following:
a. A controller interface 314, which receives commands and settings from the tactile stimulator controller 313 for operation of the tactile stimulator 107;
b. A motor 316, which is driven to produce the desired vibratory output; and
c. A power source 315, which might be required if the left connection 105 is wireless.

In some embodiments, the power source 315 may be provided from standard batteries. In other embodiments, the power might be provided from a rechargeable battery, such as a Li—Po battery or other types of rechargeable batteries.

In some embodiments, the right tactile stimulator 108 includes, but is not limited to, the following:
a. A controller interface 317, which receives commands and settings from the tactile stimulator controller 313 for operation of the tactile stimulator 108;
b. A motor 319, which is driven to produce the desired vibratory output; and
c. A power source 318, which might be required if the right connection 106 is wireless.

In some embodiments, the power source 318 may be provided from standard batteries. In other embodiments, the power might be provided from a rechargeable battery, such as a Li—Po battery or other types of rechargeable batteries.

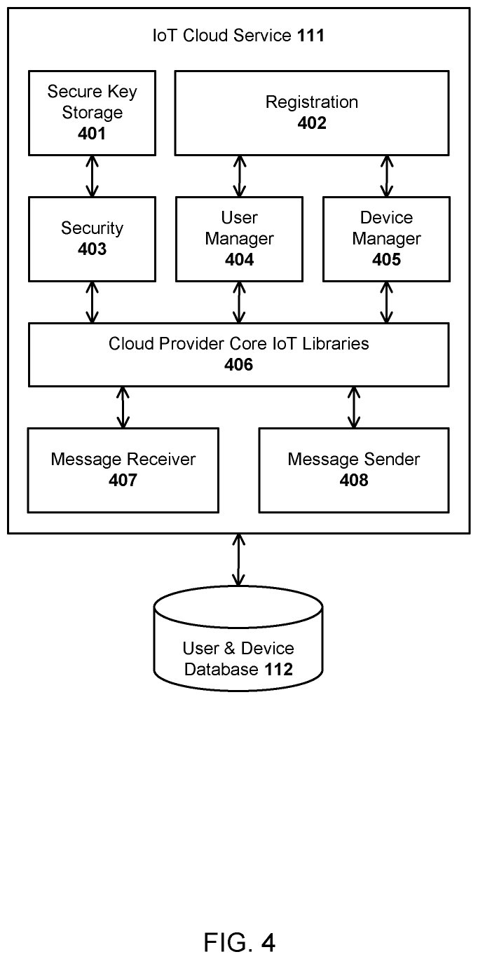

FIG. 4 illustrates the components of one embodiment of the IoT cloud service 111. These components may include but are not limited to the following:
a. A security module 403 and a mechanism for secure key storage 401;
b. A registration module 402, a user manager 404, and a device manager 405, which may be used to keep track of registered users (therapists) and their associated devices in the user and device database 112;
c. The cloud provider's core IoT libraries 406, which may be named differently on different cloud provider's platforms; and
d. The message receiver 407 and the message sender 408, which may be used to receive, send, and process messages between the therapist device 103 and the client device 104.

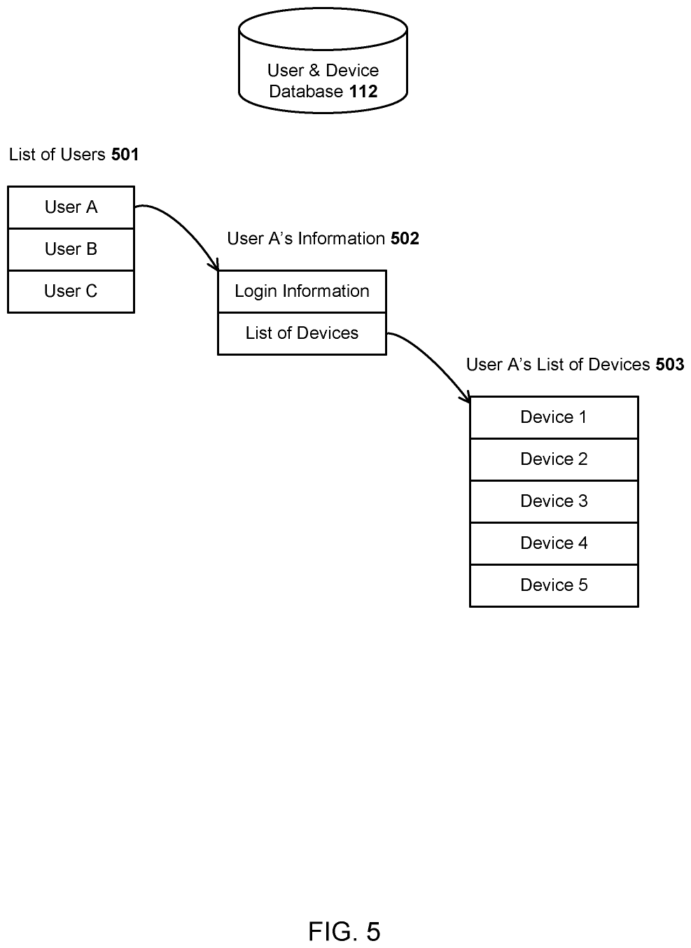

FIG. 5 shows an example of the user and device information that may be stored in the user and device database 112 in one embodiment of the invention. The structure of the data must support the following constraints.
a. A therapist may have more than one therapist IoT device 103. For example, one device 103 might be used at the therapist's office, and an additional device 103 might be used at the therapist's home;
b. A therapist may have multiple client IoT devices 104, one or more per client; and
c. The therapist must be able to select the desired client IoT device 104 for each session. The input Sensors 213 of the therapist IoT device 103 provide the means for this.

The list of users 501 refers to a list of therapists who have registered their devices with the IoT cloud service 111.

For each user in the list 501, their login information and list of devices is maintained by the cloud service 111, as shown in the example User A's information 502.

The user's list of devices, as shown in the example User A's list of devices 503, includes all the devices registered to that user. Although not shown, the list of devices includes both the user's therapist devices 103 and their client devices 104. Also not shown is the information which is maintained for each device, which might include but is not limited to entries for the unique identifier of the device, and the type of device 103 or 104.

The IoT cloud service 111 supports the ability to add new users (therapists), and to remove users. Additionally, the cloud service 111 supports the addition of new devices for a therapist, and the removal of devices.

It should be clear to one who is skilled in the art that the invention might have additional uses for a client, such as relaxation, help in grounding, or other therapeutic applications.

In a best mode for carrying out the invention:
a. The therapist device features an ESP32 based ultra low power processor, with an on-board Wi-Fi network interface and antenna. A small LCD display and physical input buttons are used for therapist input and display of parameters. In addition, the therapist may select an “auto-stop” feature which will automatically stop the BLS after a selected number of sets. The therapist may optionally select whether the left/right stimulations are simulated on the display. This option is provided because the visual display of BLS may be distracting for some therapists. A green indicator is shown upon starting BLS, and a red indicator is shown upon cessation of BLS. The current connection status to Wi-Fi, the cloud, and the client device are continually shown on the display. The current battery percentage remaining is also shown. Since a keyboard is not part of the device, a method is provided for the initial password setup or change, so that the device will automatically connect to the selected network without further interaction.
b. The client device features an ESP32 based ultra low power processor, with an on-board Wi-Fi network interface and antenna. A small LCD display is used to continually indicate the current connection status to Wi-Fi, the cloud, and the therapist device. The current battery percentage remaining is also shown. Since a keyboard is not part of the device, a method is provided for the initial password setup or change, so that the device will automatically connect to the selected network without further interaction. There is a separate Bluetooth Low Energy (BLE) module that is used for the connection to the two BLE-enabled tactile stimulators. A program broadcasts messages over BLE to the two stimulators, which monitor the broadcasts to determine when to produce the assigned left or right stimulation. By using a separate module for the BLE communication and the Wi-Fi communication, the client device is able to more tightly control the synchronization of the vibrations, while at the same time, monitoring the communication with the therapist device over Wi-Fi to detect dynamic changes for issuing the BLS vibrations.
c. The cloud service may use a standard messaging service, such as MQTT, provided by a commercial cloud provider. Individual therapist and client devices may be uniquely identified to the cloud provider and are only allowed to access the appropriate messaging system if they are authorized to do so.

I claim:

1. A system for providing remote interactive control and delivery of tactile bilateral stimulation, the system comprising:
an internet-enabled client device in communication with two tactile stimulators;
an internet-enabled therapist device configured to allow a user to select parameters and control the delivery of tactile BLS to the remotely located client device over a network; and
a cloud service that is configured to provide secure communication between the therapist and client devices.
